(19) United States
(12) Patent Application Publication
(10) Pub. No.: US 2026/0272803 A1
(43) Pub. Date: Sep. 17, 2026

(54) SUNSCREEN CONTAINING TEREPHTHALYLIDENE DICAMPHOR SULFONIC ACID AND GLYCOLS

(71) Applicant: BEIERSDORF AG, Hamburg (DE)

(72) Inventors: Laetitia NEUMANN, Welle (DE); David SCHLENKER, Hamburg (DE)

(21) Appl. No.: 19/469,066

(22) PCT Filed: Mar. 1, 2024
(86) PCT No.: PCT/EP2024/055385

§ 371 Date: Sep. 25, 2025

(30) Foreign Application Priority Data
Mar. 30, 2023 (DE) 102023202923.5

(51) Int. Cl. A61K 8/46; A61K 8/34; A61K 8/49; A61Q 17/04

(52) U.S. Cl. CPC A61K 8/466; A61K 8/345; A61K 8/4946; A61Q 17/04; A61K 2800/49

(57) ABSTRACT

A cosmetic preparation containing a) a salt of terephthalylidene dicamphor sulfonic acid (INCI: Terephthalylidene Dicamphor Sulfonic Acid), b) one or more glycols, c) water.

The present invention relates to a cosmetic preparation comprising a salt of terephthalylidene dicamphor sulfonic acid (INCI: Terephthalylidene Dicamphor Sulfonic Acid), one or more glycols and water.

For many years there has been an unbroken trend away from aristocratic pallor toward “healthy, athletically brown skin”. To achieve this, people expose their skin to solar radiation, since this causes pigment formation in the sense of melanin formation. However, the ultraviolet radiation of sunlight also has a harmful effect on the skin. Besides acute damage (sunburn), long-term damage such as an increased risk of developing skin cancer occurs in the case of excessive irradiation with light from the UVB range (wavelength: 280-320 nm). Excessive exposure to UVB and UVA radiation (wavelength: 320-400 nm) additionally leads to weakening of the elastic and collagen fibers in connective tissue. This leads to numerous phototoxic and photoallergic reactions, resulting in premature aging of the skin.

Therefore, to protect the skin, a range of photoprotective filter substances that can be used in cosmetic preparations have been developed. In most industrialized countries, these UVA and UVB filters are collated in the form of positive lists such as Annex 7 of the Kosmetikverordnung [German Cosmetics Ordinance].

The multiplicity of commercially available sunscreens must not, however, distract from the fact that these prior-art preparations have a range of disadvantages.

For many years there has been a constantly growing demand for UV-A filter substances. This can be attributed not least to numerous government regulations that require sunscreens to have a balanced ratio of UV-A and UV-B protection. However, the number of UV-A filters that can be used in cosmetics is very limited. Use is essentially made of oil-soluble compounds such as Butyl Methoxydibenzoylmethane or Diethylamino Hydroxybenzoyl Hexyl Benzoate.

In order to achieve particularly high UV protection, it is necessary, however, to incorporate UV-A filters into the aqueous phase. For this purpose, terephthalylidene dicamphor sulfonic acid (INCI: Terephthalylidene Dicamphor Sulfonic Acid) was developed around 30 years ago as a water-soluble UV-A filter substance. However, this substance has the disadvantage that it can be incorporated into the aqueous phase of a sunscreen only in a limited amount, since it tends to crystallize out of the aqueous phase again in the case of prolonged storage at cool temperatures (for example in winter at temperatures below 10° C.).

It was therefore the object of the present invention to develop an aqueous phase for a cosmetic preparation or an aqueous cosmetic preparation with terephthalylidene dicamphor sulfonic acid (INCI: Terephthalylidene Dicamphor Sulfonic Acid) in which the risk of this UV filter substance crystallizing out even at relatively high concentrations (i.e. greater than 0.4% by weight), in particular in cold conditions (i.e. below 10° C.), is effectively suppressed.

The object is achieved by a cosmetic preparation containing
a) a salt of terephthalylidene dicamphor sulfonic acid (INCI: Terephthalylidene Dicamphor Sulfonic Acid),
b) one or more glycols,
c) water.

The object is also achieved by the use of one or more glycols to increase the solubility of salts of terephthalylidene dicamphor sulfonic acid (INCI: Terephthalylidene Dicamphor Sulfonic Acid) in the aqueous phase of cosmetic preparations.

Within the scope of the present invention, glycols are defined exclusively as compounds that have 2 hydroxyl groups (OH groups). By definition, glycerin does not count as a glycol within the scope of the present invention.

Furthermore, within the scope of the present disclosure, designations as “according to the invention”, “advantageous according to the invention” etc. always relate to the preparation according to the invention, the use according to the invention and the process according to the invention (see below).

If the preparation according to the invention is an aqueous preparation, then the concentration specification “total weight of the aqueous phase” applies to the entire preparation.

It is advantageous according to the invention if the triethanolamine salt is used as the salt of terephthalylidene dicamphor sulfonic acid.

It is also advantageous in the context of the present invention if the aqueous phase comprises from 0.5% to 3% by weight of terephthalylidene dicamphor sulfonic acid, based on the total weight of the aqueous phase. The concentration range of 2% to 3% by weight of terephthalylidene dicamphor sulfonic acid, based on the total weight of the aqueous phase, is preferred here according to the invention.

Embodiments of the present invention that are advantageous according to the invention are characterized in that as glycols one or more compounds are selected from the group of propane-1,2-diol, propane-1,3-diol and butane-1,3-diol. Propane-1,2-diol and propane-1,3-diol are preferred according to the invention, with propane-1,3-diol being particularly preferred according to the invention.

In addition, it is very particularly preferred according to the invention if the glycols that are advantageous according to the invention are additionally combined with hexane-1,2-diol.

It is advantageous according to the invention if the preparation comprises the glycols in a total concentration of 1% to 10% by weight, based on the total weight of the aqueous phase. The concentration range of 4% to 8% by weight of glycols, based on the total weight of the aqueous phase, is preferred here according to the invention.

If the preparation according to the invention comprises a combination of glycols that are advantageous according to the invention with hexane-1,2-diol, then the use concentration that is preferred according to the invention for hexane-1,2-diol is 0.2% to 0.5% by weight, based on the total weight of the aqueous phase.

Embodiments that are particularly advantageous according to the invention are obtained when the preparation additionally comprises glycerin.

In such a case, the embodiments of the present invention that are advantageous according to the invention are characterized in that the preparation comprises glycerin in a concentration of 1% to 10% by weight, based on the total weight of the aqueous phase.

It is also preferred according to the invention if the weight ratio of the total amount of glycols to glycerin is from 1:1 to 2:1.

With respect to the weight ratio of terephthalylidene dicamphor sulfonic acid to the total amount of glycols, it is advantageous according to the invention if the weight ratio of terephthalylidene dicamphor sulfonic acid to the total amount of glycols is from 1:1 to 1:4.

In order to ensure complete protection against the UV rays of sunlight, it is necessary for the preparation to comprise further UV filter substances.

It has proven to be particularly advantageous according to the invention here if the preparation comprises a salt of disodium phenyl dibenzimidazole tetrasulfonate (INCI: Disodium Phenyl Dibenzimidazole Tetrasulfonate) and/or 2-phenylbenzimidazole-5-sulfonic acid salts.

If the preparation according to the invention comprises disodium phenyl dibenzimidazole tetrasulfonate (INCI: Disodium Phenyl Dibenzimidazole Tetrasulfonate), then the embodiments that are advantageous according to the invention are characterized in that the preparation comprises disodium phenyl dibenzimidazole tetrasulfonate (INCI: Disodium Phenyl Dibenzimidazole Tetrasulfonate) in a concentration of 0.5% to 3% by weight, based on the total weight of the aqueous phase.

Furthermore, in such a case it is advantageous according to the invention if the triethanolamine salt is used as the salt of disodium phenyl dibenzimidazole tetrasulfonate (INCI: Disodium Phenyl Dibenzimidazole Tetrasulfonate).

If the preparation according to the invention comprises 2-phenylbenzimidazole-5-sulfonic acid salts, then it is advantageous according to the invention if the preparation comprises 2-phenylbenzimidazole-5-sulfonic acid in a concentration of 1% to 5% by weight, based on the total weight of the aqueous phase.

Furthermore, in such a case it is advantageous according to the invention if the sodium salt is used as the salt of 2-phenylbenzimidazole-5-sulfonic acid.

Further embodiments of the present invention that are advantageous according to the invention are characterized in that the preparation comprises ethanol.

If the aqueous phase of the preparation comprises ethanol, then this is preferably used according to the invention in a concentration of 2% to 15% by weight, based on the total weight of the aqueous phase.

Furthermore, it is advantageous in the context of the present invention if the preparation comprises ethylhexylglycerin.

The use concentration that is advantageous according to the invention for ethylhexylglycerin here is from 0.1% to 0.3% by weight, based on the total weight of the aqueous phase.

It is also advantageous in the context of the present invention if the preparation comprises one or more compounds selected from the group of xanthan gum (INCI: Xanthan Gum), welan gum (INCI: Welan Gum), gellan gum (INCI: Gellan Gum), sclerotium gum (INCI: Scerotium Gum), cellulose and/or cellulose derivatives.

It is an embodiment of the present invention that is particularly advantageous according to the invention if the preparation does not comprise a lipophilic phase, but rather is an aqueous preparation. According to the invention, this preferably has a sun protection factor of greater than/equal to 15. In such a case, perfumes are not counted as part of the lipophilic phase.

According to the invention, the preparation according to the invention advantageously comprises film formers. In the context of the present invention, film formers are substances of varying composition that are characterized by the following property: if a film former is dissolved in water or other suitable solvents and the solution is then applied to the skin, then, after the solvent has evaporated, it forms a film that essentially serves to fix the light filters on the skin and thus to increase the water resistance of the product.

It is advantageous according to the invention if the preparation according to the invention comprises maize starch, maltodextrin and/or pullulan. According to the invention, these compounds may act as film formers.

Last but not least, the invention provides a process for producing a preparation, which is characterized in that terephthalylidene dicamphor sulfonic acid (INCI: Terephthalylidene Dicamphor Sulfonic Acid) and optionally disodium phenyl dibenzimidazole tetrasulfonate (INCI: Disodium Phenyl Dibenzimidazole Tetrasulfonate) are first added to water while stirring and dissolved with the aid of triethanolamine, then optionally 2-phenylbenzimidazole-5-sulfonic acid is added while stirring and dissolved with the aid of NaOH, then NaOH is used to establish a pH of at least 6.4, and in a further step the glycol is added while stirring and, after the addition of glycol, optionally further constituents are added.

### Comparative Experiment

An aqueous solution of
3% by weight of terephthalylidene dicamphor sulfonic acid (9% by weight of a 33% aqueous solution)
3% by weight of 2-phenylbenzimidazole-5-sulfonic acid and
2% by weight of disodium phenyl dibenzimidazole tetrasulfonate
was produced, which was adjusted to a pH of 7.5 using 2.5% by weight triethanolamine solution.
   4 Variants were Produced from this Clear Solution:
1 without further additives,
with 10% by weight of glycerin,
with 10% by weight of propane-1,3-diol and
with a mixture of 5% by weight of glycerin and 5% by weight of propane-1,3-diol.

Samples from all variants were stored at different temperatures (−10° C., −6° C., 20° C.) for 5 days, then the amount of precipitate formed was determined optically and the precipitate itself was analyzed microscopically. The following result was obtained at all storage temperatures:
Without further additives: very large amount of precipitate.
With 10% by weight of glycerin: large amount of precipitate.
With 10% by weight of propane-1,3-diol: little precipitate.
With a mixture of 5% by weight of glycerin and 5% by weight of propane-1,3-diol: no precipitate.

The precipitates were always terephthalylidene dicamphor sulfonic acid.

### EXAMPLES

The examples that follow are intended to illustrate the present invention without limiting it. Unless stated otherwise, all amounts, proportions and percentages are based on the weight and the total amount or on the total weight of the preparations.

| | Ex 1 | Ex 2 | Ex 3 | Ex 4 | Ex 5 | Ex 6 | Ex 7 | Ex 8 | Ex 9 | Ex 10 |
|---|---|---|---|---|---|---|---|---|---|---|
| Disodium Phenyl | 2 | 1 | 2 | 1 | 2 | 2 | 2 | 0 | 2 | 2 |
| Dibenzimidazole | | | | | | | | | | |
| Tetrasulfonate | | | | | | | | | | |
| Phenylbenzimidazole | 4 | 3 | 3 | 2 | 4 | 3 | 3 | 2 | 3 | 3 |
| Sulfonic Acid | | | | | | | | | | |
| Terephthalylidene | 3 | 3 | 3 | 3 | 2.5 | 2.5 | 2 | 3 | 3 | 2.5 |
| Dicamphor | | | | | | | | | | |
| Sulfonic Acid | | | | | | | | | | |
| Glycerin | 10 | 0 | 5 | 0 | 2 | 0 | 2 | 2 | 5 | 5 |
| Propylene Glycol | 0 | 10 | 5 | 0 | 7 | 0 | 3.5 | 8 | 0 | 5 |
| Butylene Glycol | 0 | 0 | 0 | 10 | 0 | 10 | 5 | 0 | 0 | 0 |
| Triethanolamine | 2.5 | 2.5 | 2.5 | 2.5 | 2.5 | 2.5 | 2.5 | 2.5 | 2.5 | 2.5 |
| Sodium Hydroxide | 0.8 | 0.8 | 0.8 | 0.8 | 0.8 | 0.8 | 0.8 | 0.8 | 0.8 | 0.8 |
| Xanthan Gum | 1 | 2 | 1 | 1 | 2 | 1 | 1 | 0.5 | 1 | 2 |
| Ethylhexylglycerin | 0.2 | 0.2 | 0.2 | 0 | 0.1 | 0.2 | 0.2 | 0.2 | 0.2 | 0.2 |
| Trisodium EDTA | 0.5 | 0.5 | 0.5 | 0.2 | 0.5 | 1 | 0.5 | 0.7 | 0.5 | 0.5 |
| Phenoxetol | 0.3 | 0.5 | 0.8 | 0 | 0.2 | 0.5 | 0.5 | 0.5 | 0.5 | 0.5 |
| Hexane-1,2-diol | 0.35 | 0.25 | 0.35 | 0 | 0.15 | 0.25 | 0.35 | 0.25 | 0.25 | 0.25 |
| Ethanol | 0 | 0 | 0 | 15 | 5 | 5 | 0 | 0 | 0 | 5 |
| Pullulan | 0 | 0 | 0 | 0 | 0.2 | 0 | 0 | 0 | 0.5 | 0 |
| Maize Starch | 0 | 0 | 0 | 0 | 0 | 0 | 0 | 0 | 0 | 0.1 |
| Aqua | Add | Add | Add | Add | Add | Add | Add | Add | Add | Add |
| | to | to | to | to | to | to | to | to | to | to |
| | 100 | 100 | 100 | 100 | 100 | 100 | 100 | 100 | 100 | 100 |

1.-21. (canceled)

22. A cosmetic preparation, wherein the preparation comprises:
(a) one or more salts of terephthalylidene dicamphor sulfonic acid,
(b) one or more glycols,
(c) water.

23. The preparation of claim 22, wherein (a) comprises a triethanolamine salt of terephthalylidene dicamphor sulfonic acid.

24. The preparation of claim 22, wherein an aqueous phase of the preparation comprises from 0.5% to 3% by weight of terephthalylidene dicamphor sulfonic acid, based on a total weight of the aqueous phase.

25. The preparation of claim 22, wherein (b) comprises one or more of propane-1,2-diol, propane-1,3-diol and butane-1,3-diol.

26. The preparation of claim 22, wherein the preparation further comprises glycerin.

27. The preparation of claim 22, wherein the preparation comprises (b) in a total concentration of from 1% to 10% by weight, based on a total weight of the aqueous phase.

28. The preparation of claim 22, wherein 26, wherein the preparation comprises glycerin in a concentration of from 1% to 10% by weight, based on a total weight of an aqueous phase.

29. The preparation of claim 26, wherein a weight ratio of (b) to glycerin is from 1:1 to 2:1.

30. The preparation of claim 22, wherein a weight ratio of (a) to (b) is from 1:1 to 1:4.

31. The preparation of claim 22, wherein (a) comprises a disodium salt of phenyl dibenzimidazole tetrasulfonate and/or one or more salts of 2-phenylbenzimidazole-5-sulfonic acid.

32. The preparation of claim 22, wherein the preparation comprises disodium phenyl dibenzimidazole tetrasulfonate in a concentration of from 0.5% to 3% by weight, based on a total weight of an aqueous phase.

33. The preparation of claim 23, wherein the triethanolamine salt comprises disodium phenyl dibenzimidazole tetrasulfonate.

34. The preparation of claim 22, wherein the preparation comprises 2-phenylbenzimidazole-5-sulfonic acid in a concentration of 1% to 5% by weight, based on the total weight of the aqueous phase.

35. The preparation of claim 22, wherein (a) comprises a sodium salt 2-phenylbenzimidazole-5-sulfonic acid.

36. The preparation of claim 22, wherein the preparation further comprises ethanol.

37. The preparation of claim 22, wherein the preparation further comprises ethylhexylglycerin.

38. The preparation of claim 22, wherein the preparation does not comprise a lipophilic phase, but rather is an aqueous preparation.

39. The preparation of claim 22, wherein the preparation further comprises one or more of xanthan gum, welan gum, gellan gum, sclerotium gum, cellulose, a cellulose derivative and/or one or more of maize starch, maltodextrin, pullulan.

40. A method of increasing the solubility of a salt of terephthalylidene dicamphor sulfonic acid in an aqueous phase of a cosmetic preparation, wherein the method comprises incorporating in the cosmetic preparation one or more glycols.

41. A process for producing the preparation of claim 22, wherein the method comprises adding terephthalylidene dicamphor sulfonic acid and optionally disodium phenyl dibenzimidazole tetrasulfonate to water while stirring and dissolving with the aid of triethanolamine, then optionally adding 2-phenylbenzimidazole-5-sulfonic acid while stirring and dissolving with the aid of NaOH, then establishing a pH of at least 6.4 with NaOH, then adding (b) while stirring and then optionally adding further constituents of the preparation.
